(19) United States
(12) Patent Application Publication
(10) Pub. No.: US 2016/0245784 A1
(43) Pub. Date: Aug. 25, 2016

(54) AIR QUALITY SENSING MODULE AND ALGORITHM

(71) Applicant: Alen Corporation, Austin, TX (US)

(72) Inventors: Jason MATOCHA, Pflugerville, TX (US); Bailey Briscoe JONES, Austin, TX (US); Suppawat KOSUMSUPPAMALA, Austin, TX (US); Luke JOHNSTON, Austin, TX (US)

(73) Assignee: Alen Corporation, Austin, TX (US)

(21) Appl. No.: 15/051,412

(22) Filed: Feb. 23, 2016

Related U.S. Application Data
(60) Provisional application No. 62/119,423, filed on Feb. 23, 2015.

(51) Int. Cl. G01N 33/00; G01N 1/22

(52) U.S. Cl. CPC G01N 33/0062; G01N 33/0075; G01N 1/2247; G01N 2033/0068; G01N 2001/227

(57) ABSTRACT

A control system in an air monitor is programmed to selectively direct air from more than one source over a sensor and various algorithms are then used to provide feedback regarding the difference between the particulate readings at each of the sources. In one embodiment, a data curve for a reading from one air source during a first time period is overlaid with a line which is linearly extrapolated beyond the end of that time period and the data curve for a reading from a second air source during an immediately subsequent second time period is overlaid with a line which is linearly extrapolated beyond the start of that time period. The difference in the contaminant level between the extrapolated lines at the end of the first time period and the beginning of the second time period approximates the difference in contaminant content between the two air sources.

### PRIORITY STATEMENT UNDER 35 U.S.C. §119 & 37 C.F.R. §1.78

This non-provisional application claims priority based upon prior U.S. Provisional Patent Application Ser. No. 62/119,423 filed Feb. 23, 2015 in the names of Jason Matocha, Bailey Briscoe Jones, and Suppawat Kosumsuppamala entitled “AIR QUALITY SENSING MODULE AND ALGORITHM,” the disclosure of which is incorporated herein in its entirety by reference as if fully set forth herein.

### BACKGROUND OF THE INVENTION

Air quality may be evaluated by measuring the concentrations of particulate matter, volatile organic compounds (VOCs), or other constituent components in the air which serve as indicators of overall quality. VOCs are a major component of air pollution and the presence of VOCs is indicative of poor indoor and outdoor air quality. VOCs emanate from many sources such as the operation of internal combustion engines, solvents, paints, and the off-gassing of construction materials.

Recent advances in VOC sensing technology allows for the production of inexpensive sensor components using metal oxide semiconductors and other materials and methods. Such inexpensive sensors are useful for detecting the presence of VOCs, but quantitative measurements are affected by variation between sensors and many environmental conditions such as temperature, air currents, and physical location of the sensor within an inhomogeneous air volume. These factors make it difficult to make a meaningful comparison between the VOC quantity reported from different source locations using different sensors.

For example, as air laden with VOCs passes through a VOC filter, the VOC concentration at the exit will be lower than the VOC concentration at the inlet by the amount of VOC that was absorbed or retained by the filter. However, concentration values reported from a sensor at the inlet and a sensor at the exit will not often reflect the true relative condition of the air before and after the filter.

It is desirable, therefore to have an air monitor and purification system and method having a control system in communication with an air quality monitor, such as a particle counter, and also in communication with a single sensor, wherein the sensor compares air quality entering and leaving the system, thereby providing valuable feedback regarding the efficiency of the filter, the effectiveness of the system, and related information.

### SUMMARY

In various embodiments, an air monitor of the present invention includes a contaminant or particulate sensor that detects the amount of particulate in the air, a control system programmed to selectively direct air from two or more sources to pass over the sensor, and various algorithms that are used to provide feedback regarding the difference between the quality of air, measured by the particulate readings, at each of the more than one sources.

In one embodiment, air entering a filtration system and air exiting a filtration system may be alternatively passed over a single sensor. Particulate levels from each of the air sources are analyzed. The quality of the air entering a filter during a first time period is plotted along a data curve together with the quality of the air exiting the same filter during a second period which is temporally immediately adjacent to the first time period. The data curve during the first period is overlaid with a line which is linearly extrapolated beyond both the start and end of the first period and the data curve during the second period is overlaid with a line which is linearly extrapolated beyond both the start and end of the second period. The difference in the particulate level at the linear extrapolation at the end of the first period and the linear extrapolation at the beginning of the second period approximates the difference in particulate content between the particulate level in the air entering the air filter and the particulate level in the air exiting the air filter.

In another embodiment, air entering a filtration system and air exiting a filtration system may again be alternatively passed over a single sensor. Particulate levels from each of the air sources are analyzed. The quality of the air entering a filter during a first time period is plotted along a data curve together with the quality of the air exiting the same filter during a second period which is temporally immediately adjacent to the first time period. The data curve shows the particulate concentration over time, which includes the data from a first source during a first period and the data from a second source during a second period and an inflection point therebetween. In some instances, the data curve during the first period is trending downward when testing the level of particulate in the air from the first source and, when the sensor begins testing the level of particulate in the air from the second source during the second period, the upward transition at the inflection point indicates that the particulate level in the air from the second source is greater than the particulate level in the air from the first source. Conversely, if the data curve during the second period is trending downward when testing the level of particulate in the air from the second source and, when the sensor again begins testing the level of particulate in the air from the first source during the inflection point turns downward, the particulate level in the air from the second source is greater than the particulate level in the air from the first source.

The foregoing has outlined rather broadly certain aspects of the present invention in order that the detailed description of the invention that follows may better be understood. Additional features and advantages of the invention will be described hereinafter which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### DESCRIPTION OF THE DRAWINGS

For a more complete understanding of the present invention, and the advantages thereof, reference is now made to the following descriptions taken in conjunction with the accompanying drawings, in which:

FIG. 1 shows one embodiment of the air quality monitor of the present invention;

FIG. 2 shows a graph depicting the second algorithm described herein; and

FIG. 3 shows a graph depicting the third algorithm described herein.

### DESCRIPTION OF THE PREFERRED EMBODIMENTS

The present invention is directed to improved methods and systems for, among other things, air quality monitors and purifiers. The configuration and use of the presently preferred embodiments are discussed in detail below. It should be appreciated, however, that the present invention provides many applicable inventive concepts that can be embodied in a wide variety of contexts other than an air quality sensing module and algorithm. Accordingly, the specific embodiments discussed are merely illustrative of specific ways to make and use the invention, and do not limit the scope of the invention.

As previously described, there are disadvantages to using multiple sensors to determine comparative air quality at multiple locations. Preferably, a single sensor could be used to determine particulate levels at multiple locations, thereby eliminating many of the variables that typically prevent accurate comparisons. As presented herein in various embodiments of the present invention, rather than using multiple sensors to determine comparative air quality at different source locations, air is diverted from different source locations to a single sensor. As the data from the sensor is collected, various control algorithms may be used to determine the air relative quality at the various air sources during separate, yet contiguous, time periods. An added advantage is that the use of one sensor is less costly that using multiple sensors.

If the particulate concentrations in the air being measured remain constant for a sufficient time period and a single sensor is alternating detection of two or more air source locations over comparatively substantially shorter time periods, then the average concentration values for each source location change very little and can be easily compared. In other words, for particulate concentrations that are not rapidly changing over time, a single sensor can provide meaningful results simply by alternating the source of the air location and comparing the average values from each source over time.

However, in many practical applications, particulate concentrations fluctuate with time rendering the process described above ineffective. Therefore, embodiments of the present invention provide alternative systems and methods for determining the relative particulate concentration between two or more air source locations.

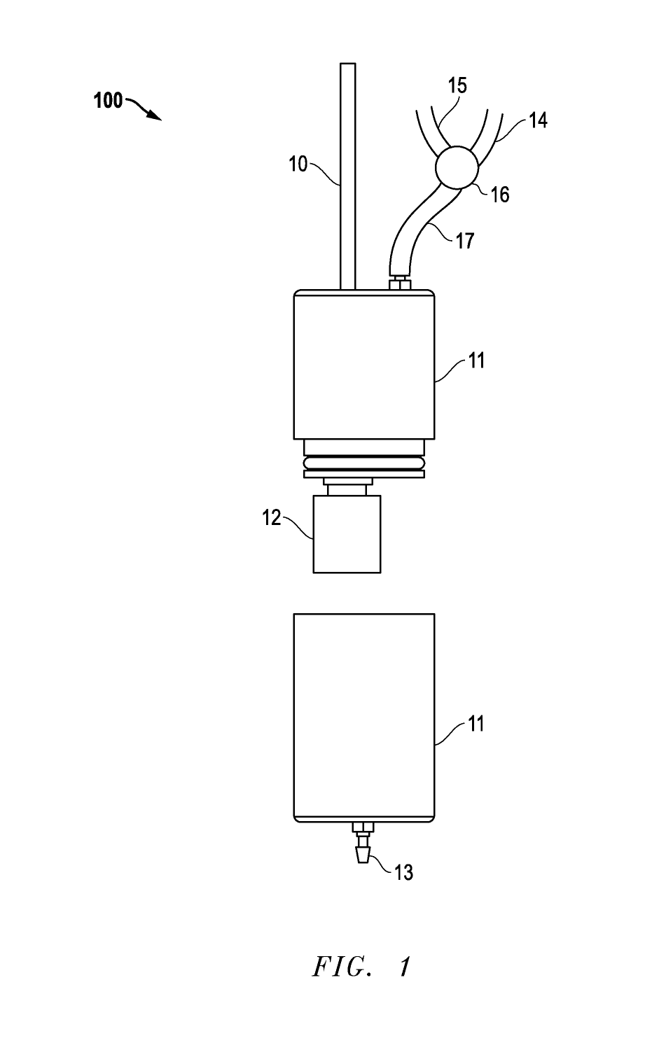

Referring now to FIG. 1 showing an air quality module 100 comprised of a sensor 12 inside of an air-tight chamber 11. The sensor 12 may be a VOC sensor or, in other embodiments, may be a particulate sensor, or other sensor that measure constituent components of air or other gases. In various descriptions of the embodiments described herein, particulate levels and contaminant levels are used interchangeably. Air from a first source is directed through a tube 14 to valve 16 and air from a second source is directed through tube 15 to valve 16. Air from a first source and air from a second source enter valve 16 which selectively directs the air into the chamber 11 through tube 17. Air leaves the chamber 11 through an exhaust port 13.

The static pressure at the exhaust port 13 is less than that of the port providing air from the first source and the port providing air from the second source, so that air is induced to flow through the chamber at a steady rate. The valve 16 is controlled to engage at intervals, thereby providing air from the various sources to the chamber 11. The sensor 12 reads and sends data through a data line 10 to a processing unit. The data will be analyzed by the processing unit using an algorithm to compare the quality of air from a first source to the quality of air from a second source. That data stream combined with the time points of valve 16 engagement will be analyzed by one of three algorithms.

A first algorithm processes the data received from the sensor 12 by simply comparing the average reading from a first source during a first reading period with the average reading from a second source during a second reading period. As described above, this comparison, although simple and convenient, provides useful information because, among other things, the data from both readings is taken from a single sensor, thereby eliminating any error resulting in defective calibration between sensors or individual sensor deterioration over time.

A second algorithm processes the data received from the sensor 12 by linearly extrapolating the discrete curve during various reading periods so that the extrapolations overlap in time with an adjacent reading period. The difference in relative quality between air samples during that overlap period is then calculated.

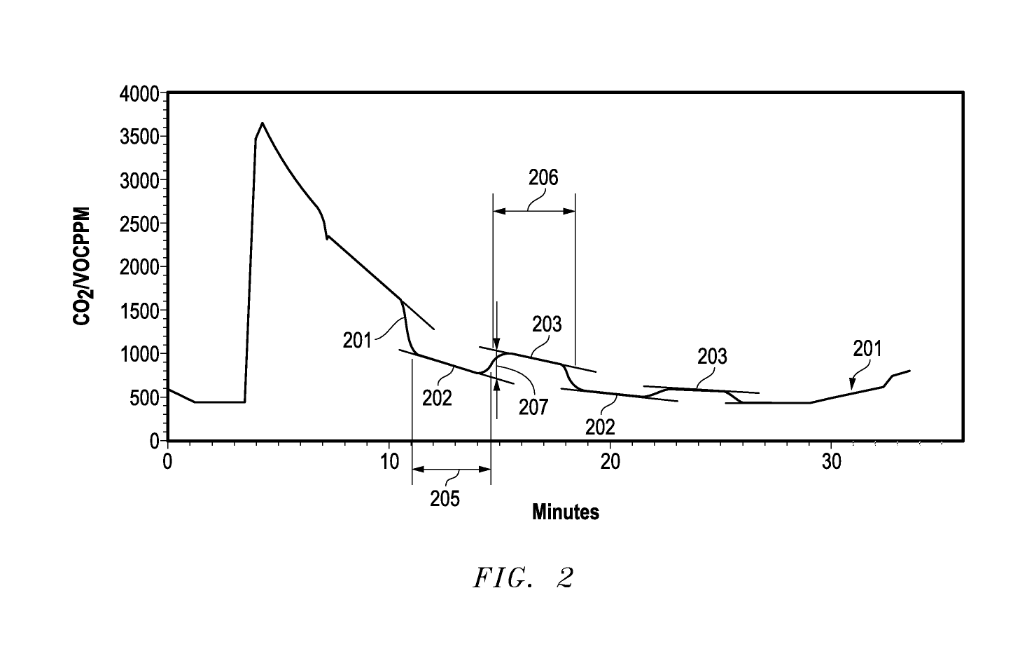

For example, FIG. 2 shows the data curve 201 in which the contaminant concentration in parts per million is plotted against time in minutes. After approximately 10 minutes, the valve 16 allows air from a first source to flow through tube 17 to access the sensor 12. After approximately 4 minutes, the valve 16 stops air from a first source from flowing through tube 17 and allows air from a second source to begin flowing through tube 17 to sensor 12. The pattern continues, with valve 16 periodically stopping air from one source to access sensor 12 while allowing air from another source to access sensor 12.

As the data received from sensor 12 is plotted over time, the data curve 201 shows the contaminant concentration over time, which includes the data from a first source during a first period 205 and the data from a second source during a second period 206. Using the second algorithm, the data curve 201 during the first period 205 is overlaid with a line 202 which is linearly extrapolated beyond both the start and end of the first period 205 and the data curve 201 during the second period 206 is overlaid with a line 203 which is linearly extrapolated beyond both the start and end of the second period 206. The difference 207 in the contaminant level at the linear extrapolation at the end of the first period 205 and the linear extrapolation at the beginning of the second period 206 approximates the difference in contaminant content between the contaminant level in the air at the first source and the contaminant level in the air at the second source.

While the second algorithm may be exceptionally useful in those instances in which the quantitative difference between the contaminant level in different air sources is required, there are other instances in which it is simply important to determine whether the contaminant level in one location is “greater than” or “less than” the contaminant level at one or more other locations. In these instances, analyzing the data curve in the transition period after switching from the data representing the contaminant level in air from a first source to the data representing the contaminant level in air from a second source provides relevant results. The slope of the best-fit line in the transition period compared with the slope immediately preceding the transition period reveals if the second source location concentration is greater than or less than the first.

A third algorithm allows for rapid switching between air sources, because the data curve is not required to stabilize prior to switching the air passing over the sensor from one source to another. Rather, a change in slope of the data curve is calculated before and after switching the air source. If the slope increases, the second source has a greater concentration of contaminants. If the slope decreases, the second source has a lower concentration of contaminants. This method is especially applicable for determining the performance of a contaminant filter.

For example, if a first air sensor is positioned at the location at which air enters an air filtration device and a second air sensor is positioned at the location at which air exits the same air filtration device, it is possible to determine the filter effectiveness and, for example when the filter stops working because it has become saturated.

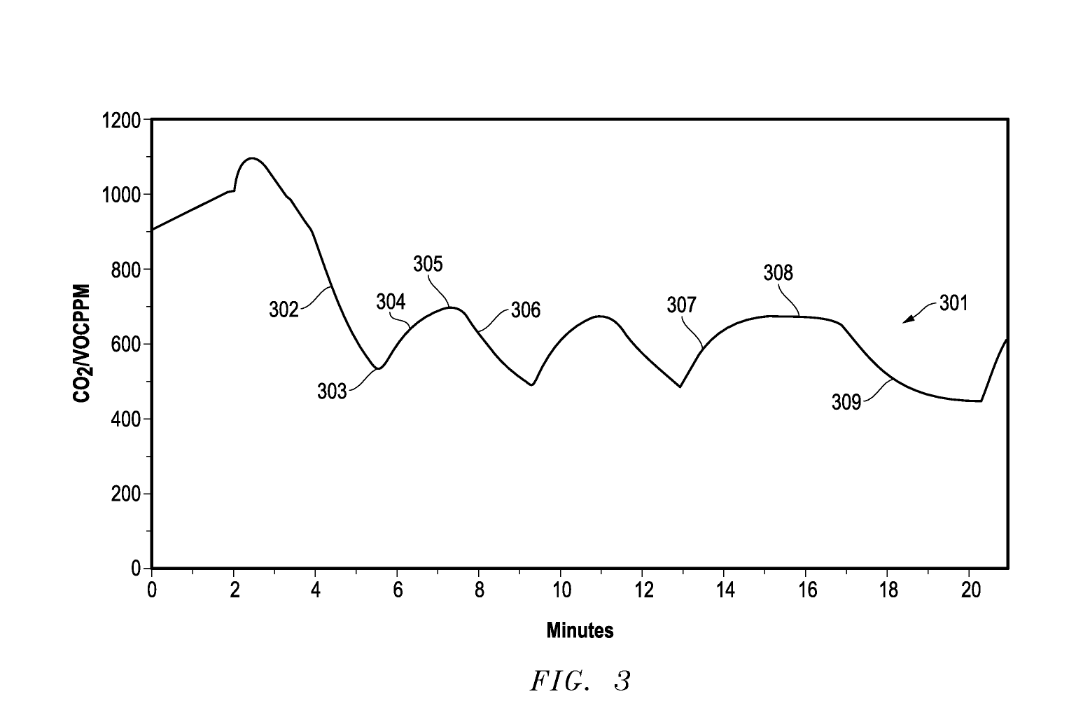

Referring now to FIG. 3 with reference back to FIG. 1, the data curve 301 shows the contaminant concentration in parts per million is plotted against time in minutes. After approximately 2 minutes, the valve 16 allows air from a first source to flow through tube 17 to access the sensor 12. After approximately 5 minutes, the valve 16 stops air from a first source from flowing through tube 17 and allows air from a second source to begin flowing through tube 17 to sensor 12. Once again, the pattern continues, with valve 16 periodically stopping air from one source to access sensor 12 while allowing air from another source to access sensor 12.

As the data received from sensor 12 is plotted over time, the data curve 301 shows the contaminant concentration over time, which includes the data from a first source during a first period 302 and the data from a second source during a second period 304 and an inflection point 303 therebetween. Using the third algorithm, the data curve 301 during the first period 302 is trending downward when testing the level of contaminant in the air from the first source and, when the sensor begins testing the level of contaminant in the air from the second source during the second period 304, the upward transition at the inflection point 303 indicates that the contaminant level in the air from the second source is greater than the contaminant level in the air from the first source. Conversely, the data curve 301 during the second period 304 is trending downward when testing the level of contaminant in the air from the second source and, when the sensor again begins testing the level of contaminant in the air from the first source during the third period 306, the downward transition at the inflection point 305 confirms that the contaminant level in the air from the second source is greater than the contaminant level in the air from the first source.

As the different sources of air continue to cycle over the sensor, the directional changes at the inflection points provide information regarding the relative contaminant level in air from the first source and the air from the second source. Moreover, the greater the rate of change in the data curve, the greater the difference in the relative contamination levels between the two sources. By way of example, the difference in the slope of the data curve 301 during the first period 302 and the slope of the data curve 301 during the second period 304 creates an abrupt change in direction of the data curve 301 at inflection point 303. Conversely, the difference in the slope of the data curve 301 during the sixth period 307 and the slope of the data curve 301 during the seventh period 309 creates a comparatively very gradual change in direction of the data curve 301 at inflection point 308. Therefore, the degree of change in the relative slope at an inflection point provides useful information regarding the comparative air quality at two different locations.

Again by way of example, and again referring back to FIG. 1 and FIG. 2, an air filter may be configured air with an uptake tube 14 at the point at which air enters the filter and an uptake tube 15 at a point at which air exits the filter. Both the uptake tube 14 and the uptake tube 15 are fluidly connected to a valve 16 which is fluidly coupled to and configured to selectively provide air from either uptake tube 14 or uptake tube 15 to sensor 12.

As the data received from sensor 12 is plotted over time, the data curve 201 shows the contaminant concentration, which includes the data from the air entering the filter during a first period 205 and the data from the air exiting the filter during a second period 206. Using the second algorithm described above, the data curve 201 during the first period 205 is overlaid with a line 202 which is linearly extrapolated beyond both the start and end of the first period 205 and the data curve 201 during the second period 206 is overlaid with a line 203 which is linearly extrapolated beyond both the start and end of the second period 206. The difference 207 in the contaminant level at the linear extrapolation at the end of the first period 205 and the linear extrapolation at the beginning of the second period 206 approximates the difference in contaminant content between the contaminant level in the air entering the filter and the contaminant level in the air exiting the air filter. If the difference between the air entering the filter and the air leaving the filter is above a certain threshold, then an indicator may, for example, indicate that a filter needs to be changed or that a fan speed needs to be adjusted to increase the flow of air through the filter.

While the present system and method has been disclosed according to the preferred embodiment of the invention, those of ordinary skill in the art will understand that other embodiments have also been enabled. Even though the foregoing discussion has focused on particular embodiments, it is understood that other configurations are contemplated. In particular, even though the expressions “in one embodiment” or “in another embodiment” are used herein, these phrases are meant to generally reference embodiment possibilities and are not intended to limit the invention to those particular embodiment configurations. These terms may reference the same or different embodiments, and unless indicated otherwise, are combinable into aggregate embodiments. The terms “a”, “an” and “the” mean “one or more” unless expressly specified otherwise. The term “connected” means “communicatively connected” unless otherwise defined.

When a single embodiment is described herein, it will be readily apparent that more than one embodiment may be used in place of a single embodiment. Similarly, where more than one embodiment is described herein, it will be readily apparent that a single embodiment may be substituted for that one device.

In light of the wide variety of air quality monitors and purifiers known in the art, the detailed embodiments are intended to be illustrative only and should not be taken as limiting the scope of the invention. Rather, what is claimed as the invention is all such modifications as may come within the spirit and scope of the following claims and equivalents thereto.

None of the description in this specification should be read as implying that any particular element, step or function is an essential element which must be included in the claim scope. The scope of the patented subject matter is defined only by the allowed claims and their equivalents. Unless explicitly recited, other aspects of the present invention as described in this specification do not limit the scope of the claims.

What is claimed is:

1. An air quality monitor comprising:
a sensor for determining the amount of particulate in air;
a first uptake tube configured to convey air from a first location to a valve;
a second uptake tube configured to convey air from a second location to the valve, the valve being configured to switch between providing air from the first uptake tube to the sensor during a first period of time and providing air from the second uptake tube to the sensor during a second period of time;
wherein the sensor provides to a processor a first set of data relating the particulate in the air from the first uptake tube over the first period, and a second set of data relating the particulate in the air from the second uptake tube over the second period;
the processor processing the first set of data and the second set of data to provide information regarding the quantity of particulate in the air at the first location relative to the particulate in the air at the second location.

2. The air quality monitor of claim 1, wherein the first uptake tube is located on the inlet side of an air filter and the second uptake tube is located on the exit side of the air filter.

3. The air quality monitor of claim 1, wherein the particulate is a volatile organic compound.

4. The air quality monitor of claim 1, wherein the second time period immediately follows the first time period, and wherein the processor creates a first data curve from the first set of data and a second data curve from the second set of data and overlays a first line approximating the first data curve that is extrapolated beyond the end of the first time period and overlays a second line approximating the second data curve that is extrapolated beyond the start of the second time period, and wherein the difference between the first line and the second line is used to approximate the difference between the particulate level at the first location and the particulate level at the second location.

5. The air quality monitor of claim 1, wherein the second time period immediately follows the first time period, and wherein the processor creates a first data curve from the first set of data and a second data curve from the second set of data and the processor determines the particulate in the air is greater in the second sensor if the second data curve is trending upward after the inflection point between the first data curve and the second data curve.

6. The air quality monitor of claim 1, wherein the second time period immediately follows the first time period, and wherein the processor creates a first data curve from the first set of data and a second data curve from the second set of data and, if the processor determines the particulate in the air is greater in the second sensor because the second data curve is trending upward after the inflection point between the first data curve and the second data curve, then an indicator alerts the user to take appropriate action.

7. An air filter system comprising:
a sensor for determining the amount of particulate in air;
a filter for filtering particulate from air;
a first uptake tube configured to convey air from a point at which air is entering the filter to a valve;
a second uptake tube configured to convey air from a point at which air is exiting the filter to the valve, the valve being configured to switch between providing air from the first uptake tube to the sensor during a first period of time and providing air from the second uptake tube to the sensor during a second period of time;
wherein the sensor provides to a processor a first set of data relating the particulate in the air from the first uptake tube over the first period, and a second set of data relating the particulate in the air from the second uptake tube over the second period;
the processor processing the first data and the second data to provide information regarding the quantity of particulate in the air entering the filter relative to the particulate in the air exiting the filter.

8. The filter system of claim 7, wherein the particulate is a volatile organic compound.

9. The filter system of claim 7, wherein the second time period immediately follows the first time period, and wherein the processor creates a first data curve from the first set of data and a second data curve from the second set of data and overlays a first line approximating the first data curve that is extrapolated beyond the end of the first time period and overlays a second line approximating the second data curve that is extrapolated beyond the start of the second time period, and wherein the difference between the first line and the second line is used to approximate the quantity of particulate in the air entering the filter relative to the particulate in the air exiting the filter.

10. The filter system of claim 7, wherein the second time period immediately follows the first time period, and wherein the processor creates a first data curve from the first set of data and a second data curve from the second set of data and the processor determines the particulate in the air is greater in the second sensor if the second data curve is trending upward after the inflection point between the first data curve and the second data curve.

11. The filter system of claim 7, wherein the second time period immediately follows the first time period, and wherein the processor creates a first data curve from the first set of data and a second data curve from the second set of data and, if the processor determines the particulate in the air is greater in the second sensor because the second data curve is trending upward after the inflection point between the first data curve and the second data curve, then an indicator alerts the user to replace the filter.

12. A method of monitoring air comprising:
determining the amount of particulate in air using a single sensor;
conveying air from a first location to a valve through a first uptake tube;
conveying air from a second location to a valve through a second uptake tube, the valve being configured to switch between providing air from the first uptake tube to the sensor during a first period of time and providing air from the second uptake tube to the sensor during a second period of time;
providing from the sensor to the processor a first set of data relating the particulate in the air from the first uptake tube over the first period, and a second set of data relating the particulate in the air from the second uptake tube over the second period;
processing with the processor the first set of data and the second set of data to provide information regarding the quantity of particulate in the air at the first location relative to the particulate in the air at the second location.

13. The method of monitoring air of claim 12, wherein the first uptake tube is located on the inlet side of an air filter and the second uptake tube is located on the exit side of the air filter.

14. The method of monitoring air of claim 12, wherein the particulate is a volatile organic compound.

15. The method of monitoring air of claim 12, wherein the second time period immediately follows the first time period, and wherein the processor creates a first data curve from the first set of data and a second data curve from the second set of data and overlays a first line approximating the first data curve that is extrapolated beyond the end of the first time period and overlays a second line approximating the second data curve that is extrapolated beyond the start of the second time period, and wherein the difference between the first line and the second line is used to approximate the difference between the particulate level at the first location and the particulate level at the second location.

16. The method of monitoring air of claim 12, wherein the second time period immediately follows the first time period, and wherein the processor creates a first data curve from the first set of data and a second data curve from the second set of data and the processor determines the particulate in the air is greater in the second sensor if the second data curve is trending upward after the inflection point between the first data curve and the second data curve.

17. The method of monitoring air of claim 12, wherein the second time period immediately follows the first time period, and wherein the processor creates a first data curve from the first set of data and a second data curve from the second set of data and, if the processor determines the particulate in the air is greater in the second sensor because the second data curve is trending upward after the inflection point between the first data curve and the second data curve, then an indicator alerts the user to take appropriate action.
